(19) United States
(12) Patent Application Publication
(10) Pub. No.: US 2021/0205209 A1
(43) Pub. Date: Jul. 8, 2021

(54) Composition for Topical Pain Rub and a Manufacturing Method Thereof

(71) Applicant: Alma McKay, Douglasville, GA (US)

(72) Inventor: Alma McKay, Douglasville, GA (US)

(21) Appl. No.: 16/737,879

(22) Filed: Jan. 8, 2020

(51) Int. Cl. A61K 9/00; A61K 36/534; A61K 36/28

(52) U.S. Cl. CPC A61K 9/0014; A61K 36/28; A61K 36/534

(57) ABSTRACT

A composition for topical pain rub and a manufacturing method thereof facilitates the healing process for muscle and joint pains and enhances soothing sensations. The composition includes a quantity of peppermint oil, a quantity of spearmint oil, a quantity of alcohol, a quantity of menthol oil, and a quantity of calendula oil. The composition may further include a quantity of echinacea oil.

The current application claims priority to U.S. provisional application Ser. No. 62/789,926 filed on Jan. 8, 2019.

### FIELD OF THE INVENTION

The present invention generally relates to compositions for topical pain rub. More specifically, the present invention produces a quantity of rubbing liquid solution for fast and soothing relief for pain and minor aches.

### BACKGROUND OF THE INVENTION

Pain relieving ointments is a common and necessary supplement for individuals who are active. Such individuals may play sports, exercise regularly, perform plenty of physical movement that may strain muscles or receive injuries from nearby individuals or objects. Minor injuries or aches may simply take time to heal. However, an individual does not need to suffer the entire time waiting to be healed.

The present invention is an all-natural rub for treatment of pain and minor aches. The present invention works by simply applying a quantity of rubbing liquid solution to the problem area and massage into skin. In order to further enhance the soothing effects of the present invention, once applied to the skin, soak in a bath with the quantity of rubbing liquid solution having been evenly applied to the skin. The present invention is great for treatment of a chronic condition or injury or are recovering from surgery. The present invention works to speed healing and strengthening. The present invention serves to ease individuals ranging from elite athletes to those who lead less active lifestyles.

### BRIEF DESCRIPTION OF THE DRAWINGS

FIG. 1 is a block diagram of the present invention.

FIG. 2 is a block diagram illustrating the proportions of the present invention.

FIG. 3 is a block diagram of a preferred embodiment of the present invention.

FIG. 4 is a block diagram illustrating the proportion of the preferred embodiment of the present invention.

### DETAIL DESCRIPTIONS OF THE INVENTION

All illustrations of the drawings are for the purpose of describing selected versions of the present invention and are not intended to limit the scope of the present invention.

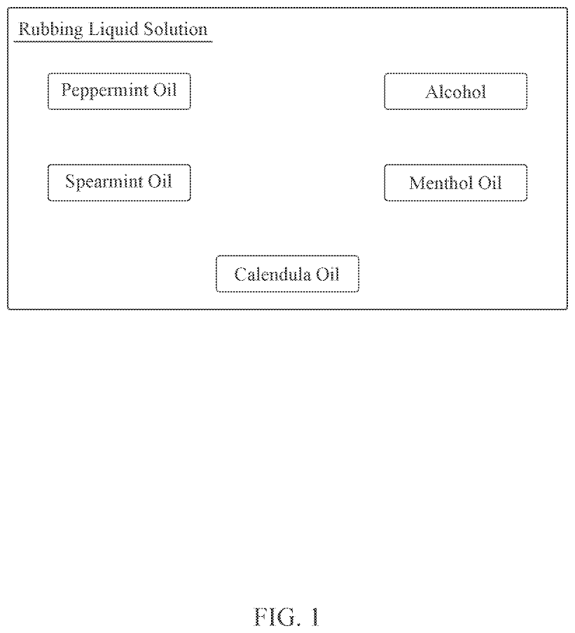

The present invention is a composition for a topical pain rub and a manufacturing method thereof. The topical pain rub is applied to the skin of an area along the body of a user that is experiencing pain. The topical pain rub may be applied as necessary. The topical pain rub facilitates the healing of injuries with muscles and joints and soothes the skin around the injury. The composition of the topical pain rub comprises a quantity of peppermint oil, a quantity of spearmint oil, a quantity of alcohol, a quantity of menthol oil, and a quantity of calendula oil, as seen in FIG. 1. The quantity of peppermint oil soothes aches by providing a cooling effect. Furthermore, the quantity of peppermint oil relieves inflammation. The quantity of spearmint oil relives muscle strains and spasms. The quantity of alcohol relieves minor skin irritations and creates a smoother and lighter solution. The quantity of alcohol also allows the quantity of peppermint oil, the quantity of spearmint, the quantity of menthol oil, and the quantity of calendula oil to easily penetrate the skin of the applied area. The quantity of menthol oil also further cools the area of injury. The quantity of calendula oil is a natural oil with anti-fungal, anti-inflammatory, and anti-bacterial properties that soothes the skin. Moreover, the quantity of calendula facilitates the healing of skin around the area of injury.

The quantity of peppermint oil, the quantity of spearmint oil, the quantity of alcohol, the quantity of menthol oil, and the quantity of calendula oil are homogeneously mixed into a rubbing liquid solution. The rubbing liquid solution therefore provides all the healing and soothing properties of each ingredient simultaneously to an injured or aching area of the body.

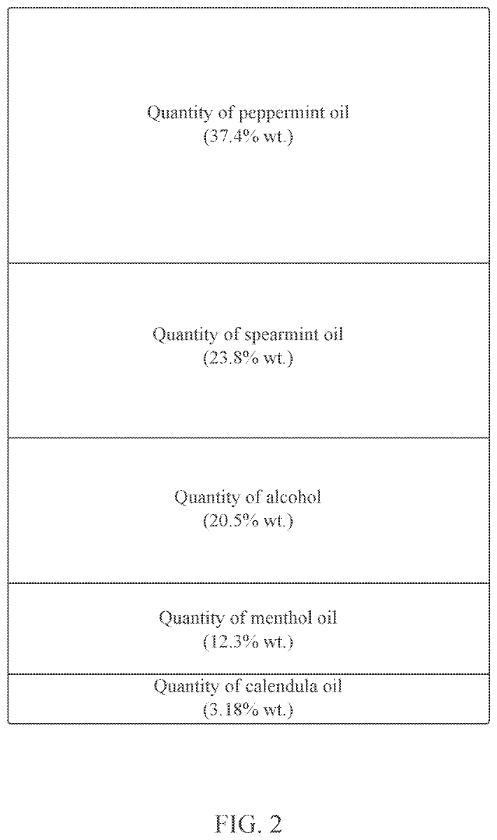

In order to properly proportion the liquid rubbing solution that facilitates the healing and soothes skin, the quantity of peppermint oil is about 37.4 percentage by weight (wt. %) of the rubbing liquid solution, seen in FIG. 2. The quantity of spearmint oil is about 23.8 wt. % of the rubbing liquid solution. The quantity of alcohol is about 20.5 wt. % of the rubbing liquid solution. The quantity of menthol oil is about 12.3 wt. % of the rubbing liquid solution. The quantity of calendula oil is about 3.18 wt. % of the rubbing liquid solution. The quantity of peppermint oil, the quantity of spearmint oil, the quantity of alcohol, the quantity of menthol oil, and the quantity of calendula oil may each vary between +/−2.5 wt. %.

In order to alternatively proportion the liquid rubbing solution that facilitates the healing and soothes skin, the quantity of peppermint oil is about 36.4 percentage by volume (vol. %) of the rubbing liquid solution at Standard Temperature and Pressure (STP). The quantity of spearmint oil is about 22.72 vol. % of the rubbing liquid solution at STP. The quantity of alcohol is about 22.72 vol. % of the rubbing solution at STP. The quantity of menthol oil is about 12.12 vol. % of the rubbing liquid solution at STP. The quantity of calendula oil is about 3.03 vol. % of the rubbing liquid solution at STP. The quantity of peppermint oil, the quantity of spearmint oil, the quantity of alcohol, the quantity of menthol oil, and the quantity of calendula oil may each vary between +/−2.5 vol. %.

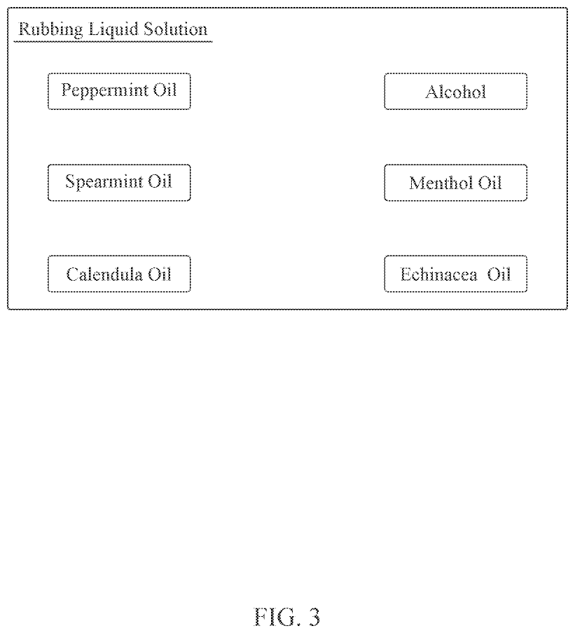

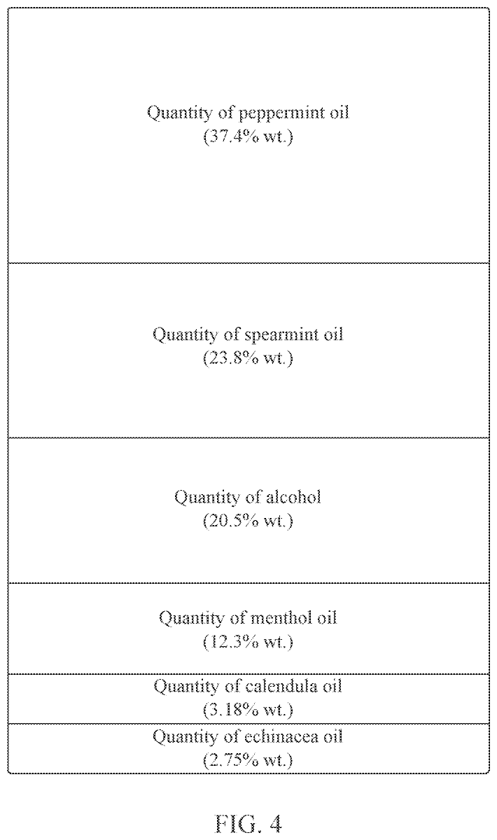

In some embodiments of the present invention, the present invention further comprises a quantity of echinacea oil, as seen in FIG. 3. The quantity of echinacea oil further enhances the anti-inflammatory properties of the rubbing liquid solution. Furthermore, the quantity of echinacea oil also aids the relief of any damage to the skin. The quantity of echinacea oil is further homogeneously mixed into the rubbing liquid solution. In order to enhance the healing and soothing effects of the rubbing liquid solution, the quantity of echinacea oil is about 2.75 wt. % of the rubbing liquid solution, seen in FIG. 4, or can alternatively be about 3.03 vol. % of the rubbing liquid solution. Similarly, the quantity of echinacea oil may vary between +/−2.5 wt. % or +/−2.5 vol. %.

The method of manufacturing the rubbing liquid solution includes the following steps that are implemented with the quantity of peppermint oil, the quantity of spearmint oil, the quantity of alcohol, the quantity of menthol oil, and the quantity of calendula oil. The amount of each ingredient is first measured. Moreover, the quantity of peppermint oil is preferably 48 fluid ounces (fl. oz.), the quantity of spearmint oil is preferably 30 fl. oz., the quantity of alcohol is preferably 30 fl. oz., the quantity of menthol oil is preferably 16 fl. oz., and the quantity of calendula oil is preferably 4 fl. oz. In some embodiments of the present invention, the quantity of echinacea is preferably 4 fl. oz. The quantity of peppermint oil, the quantity of spearmint oil, the quantity of alcohol, the quantity of menthol oil, and the quantity of calendula oil is thoroughly mixed into the liquid rubbing solution with a smooth topical sensation. The quantity of echinacea is also added and thoroughly mixed into the liquid rubbing solution in some embodiments of the present invention. The rubbing liquid solution may be topically applied to areas of aches and pain around the body.

Although the invention has been explained in relation to its preferred embodiment, it is to be understood that many other possible modifications and variations can be made without departing from the spirit and scope of the invention as hereinafter claimed.

What is claimed is:

1. A composition for topical pain rub comprises:
a quantity of peppermint oil;
a quantity of spearmint oil;
a quantity of alcohol;
a quantity of menthol oil;
a quantity of calendula oil; and,
the quantity of peppermint oil, the quantity of spearmint oil, the quantity of alcohol, the quantity of menthol oil, and the quantity of calendula oil being homogenously mixed into a rubbing liquid solution.

2. The composition for topical pain rub as claimed in claim 1 comprises:
the quantity of peppermint oil being about 37.4 percentage by weight (wt. %) of the rubbing liquid solution.

3. The composition for topical pain rub as claimed in claim 1 comprises:
the quantity of spearmint oil being about 23.8 wt. % of the rubbing liquid solution.

4. The composition for topical pain rub as claimed in claim 1 comprises:
the quantity of alcohol being about 20.5 wt. % of the rubbing liquid solution.

5. The composition for topical pain rub as claimed in claim 1 comprises:
the quantity of menthol oil being about 12.3 wt. % of the rubbing liquid solution.

6. The composition for topical pain rub as claimed in claim 1 comprises:
the quantity of calendula oil being about 3.18 wt. % of the rubbing liquid solution.

7. The composition for topical pain rub as claimed in claim 1 comprises:
a quantity of echinacea oil; and,
the quantity of echinacea oil being further homogeneously mixed into the rubbing liquid solution.

8. The composition for topical pain rub as claimed in claim 7 comprises:
the quantity of echinacea oil being about 2.75 wt. % of the rubbing liquid solution.

9. A composition for topical pain rub comprises:
a quantity of peppermint oil;
a quantity of spearmint oil;
a quantity of alcohol;
a quantity of menthol oil;
a quantity of calendula oil;
a quantity of echinacea oil; and,
the quantity of peppermint oil, the quantity of spearmint oil, the quantity of alcohol, the quantity of menthol oil, the quantity of calendula oil, and the quantity of echinacea oil being homogenously mixed into a rubbing liquid solution.

10. The composition for topical pain rub as claimed in claim 9 comprises:
the quantity of peppermint oil being about 37.4 percentage by weight (wt. %) of the rubbing liquid solution.

11. The composition for topical pain rub as claimed in claim 9 comprises:
the quantity of spearmint oil being about 23.8 wt. % of the rubbing liquid solution.

12. The composition for topical pain rub as claimed in claim 9 comprises:
the quantity of alcohol being about 20.5 wt. % of the rubbing liquid solution.

13. The composition for topical pain rub as claimed in claim 9 comprises:
the quantity of menthol oil being about 12.3 wt. % of the rubbing liquid solution.

14. The composition for topical pain rub as claimed in claim 9 comprises:
the quantity of calendula oil being about 3.18 wt. % of the rubbing liquid solution.

15. The composition for topical pain rub as claimed in claim 9 comprises:
the quantity of echinacea oil being about 2.75 wt. % of the rubbing liquid solution.

16. A composition for topical pain rub comprises:
a quantity of peppermint oil;
a quantity of spearmint oil;
a quantity of alcohol;
a quantity of menthol oil;
a quantity of calendula oil;
the quantity of peppermint oil being about 37.4 percentage by weight (wt. %) of the rubbing liquid solution.
the quantity of spearmint oil being about 23.8 wt. % of the rubbing liquid solution.
the quantity of alcohol being about 20.5 wt. % of the rubbing liquid solution.
the quantity of menthol oil being about 12.3 wt. % of the rubbing liquid solution.
the quantity of calendula oil being about 3.18 wt. % of the rubbing liquid solution; and,
the quantity of peppermint oil, the quantity of spearmint oil, the quantity of alcohol, the quantity of menthol oil, and the quantity of calendula oil being homogenously mixed into a rubbing liquid solution.

17. The composition for topical pain rub as claimed in claim 16 comprises:
a quantity of echinacea oil; and,
the quantity of echinacea oil being further homogeneously mixed into the rubbing liquid solution.

18. The composition for topical pain rub as claimed in claim 17 comprises:
the quantity of echinacea oil being about 2.75 wt. % of the rubbing liquid solution.
